(19) United States
(12) Patent Application Publication
(10) Pub. No.: US 2025/0325809 A1
(43) Pub. Date: Oct. 23, 2025

(54) SYSTEM AND METHOD FOR PREVENTION OF ATROPHY AND / OR DEEP-VEIN THROMBOSIS EDEMA (DVT)

(71) Applicant: Restorative Therapies, Inc., Nottingham, MD (US)

(72) Inventors: James Janicki, Pheonix, MD (US); Scott Simcox, Baltimore, MD (US)

(21) Appl. No.: 19/185,919

(22) Filed: Apr. 22, 2025

Related U.S. Application Data
(60) Provisional application No. 63/637,173, filed on Apr. 22, 2024.

(51) Int. Cl. A61N 1/36; A61N 1/04

(52) U.S. Cl. CPC A61N 1/36003; A61N 1/0452; A61N 1/0484; A61N 1/36031; A61N 1/36034

(57) ABSTRACT

In a first aspect, a system for prevention of permanent atrophy and/or deep-vein thrombosis (DVT) edema is presented. The system includes a cuff. The cuff includes a first electrode positioned on a front portion of the cuff. The first electrode is configured to contract a first muscle of a user. The cuff includes a second electrode positioned on a back portion of the cuff, the back portion opposite the front portion. The second electrode is configured to contract a second muscle of a user. The system includes a stimulation unit in electrical communication with the cuff. The stimulation unit includes a controller. The controller is configured to activate the first and second electrodes to simultaneously contract the first muscle of the user while providing a variable resistance to the first muscle of the user through contraction of the second muscle of the user.

### CROSS-REFERENCE TO RELATED APPLICATIONS

This application claims priority to, and the benefit of, U.S. Prov. App. No. 63/637,173, filed Apr. 22, 2024, the entirety of which is incorporated herein by reference.

### TECHNICAL FIELD

This disclosure relates to systems of Intermittent Pneumatic Compression (IPC) and electrical stimulation exercise devices and methods for prevention of permanent atrophy and/or deep-vein thrombosis (DVT) edema and rehabilitation of individuals confined to a bed.

### BACKGROUND

The result of prolonged sedentary bed rest creates physical, cognitive, and mental health impairments that compound patients' pre-existing dysfunction. Intensive care unit (ICU) acquired weakness is one of the most important and common results of critical illness, which can affect up to 70% of ICU survivors and are often long-lasting.

### SUMMARY

In a first aspect, a system for prevention of permanent atrophy and/or deep-vein thrombosis (DVT) edema is presented. The system includes a cuff. The cuff includes a first electrode positioned on a front portion of the cuff. The first electrode is configured to contract a first muscle of a user. The cuff includes a second electrode positioned on a back portion of the cuff, the back portion opposite the front portion. The second electrode is configured to contract a second muscle of a user. The system includes a controller in electrical communication with the cuff. The controller includes a stimulation unit. The stimulation unit is configured to activate the first and second electrodes to simultaneously contract the first muscle of the user while providing a variable resistance to the first muscle of the user through contraction of the second muscle of the user.

In another aspect, a method of preventing permanent atrophy and/or deep-vein thrombosis (DVT) edema is presented. The method includes placing a cuff on a leg of a user. The cuff includes a first electrode positioned on a front portion of the cuff. The first electrode is configured to contract a first muscle of the user. The cuff includes a second electrode position on a back portion of the cuff, the back portion opposite the front portion. The second electrode is configured to contract a second muscle of the user. The method includes contracting the first muscle of the user through the first electrode. The method includes contracting the second muscle of the user through the second electrode to apply a variable resistance to the first muscle of the user.

### BRIEF DESCRIPTION OF THE DRAWINGS

These and other features, aspects, and advantages of the present disclosure will become better understood with regard to the following description and accompanying drawings.

FIG. 1 illustrates a block diagram of an exemplary embodiment of a system for prevention of permanent atrophy and/or deep-vein thrombosis edema (DVT);

FIG. 2 is an illustration of a cuff;

FIG. 3 illustrates a flowchart of a method of prevention of permanent atrophy and/or DVT edema; and

FIG. 4 is a diagram of a computing device that may be implemented in systems and methods described throughout this disclosure

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

Aspects of the present disclosure can be used to prevent permanent atrophy and/or deep-vein thrombosis (DVT) edema of an individual. In some embodiments, aspects of the present disclosure include providing resistance to a first contracting leg muscle of a user by co-contracting an opposing muscle of a user. The present disclosure can be used to help rehabilitate leg muscles of patients without a need of resistance equipment. Resistance training of the present disclosure may be modified based on a patient's rehabilitation program, muscle strength progress, or other factors. The present system may be used to contract one or more muscles of a user through various ranges of motion. In some embodiments, a controller of the present system may act in a feedback loop with a sensor to provide variable resistance to a user's leg muscles.

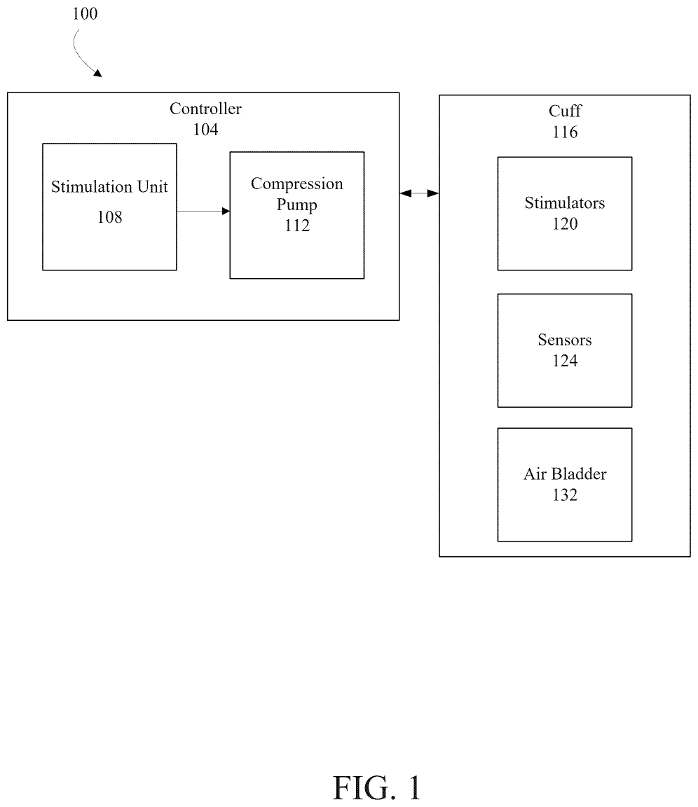

Referring now to FIG. 1, system 100 for prevention of permanent atrophy and/or DVT edema is presented. System 100 may include controller 104. Controller 104 may include one or more processors, memories, and the like. Controller 104 may include stimulation unit 108. Controller 108 may be any processing device, such as, but not limited to, microcontroller, microprocessor, system on a chip (SoC), and/or other computing devices. Controller 108 may be configured to communicate with compression pump 112, stimulators 120, sensors 124, and/or resistance device 128, such as through a wired or wireless connection.

In some embodiments, system 100 may include cuff 116. A “cuff” as used in this disclosure is a device attachable around a user's body part. Cuff 116 may at least partially encircle a limb of a user. In some embodiments, cuff 116 may be attachable to a non-limb of a user to secure itself to a limb or other body part of the user. For instance, cuff 116 may attach around a leg, arm, finger, torso, or other portion of a user. Cuff 116 may be attachable to a limb of a user through Velcro, straps, hooks, and/or other attachment devices. In some embodiments, cuff 116 may be configured to wrap around or otherwise attach to a user's leg.

Cuff 116 may include stimulators 120. “Stimulators” as used in this disclosure are any device capable of delivering a current to a body part. Stimulators 120 may include one or more electrodes. In some embodiments, stimulators 120 may be 6-channel stimulators or other types of stimulators. In some embodiments, one or more electrodes of stimulators 120 may include an electrode pad. An electrode pad may be adhesive and may deliver a current to an area of a user's body. In some embodiments, cuff 116 may have stimulators 120 placed at various areas within and/or around cuff 116. Cuff 116 may have one or more electrical connectors that may provide electrical communication from an external power source to one or more stimulators 120. For instance, cuff 116 may have an electrical connection port that be in electrical communication with one or more stimulators 120. An electrical connection port may be connectable to one or more power cables, which may be connected to stimulator unit 104. In some embodiments, cuff 116 may include about 12 stimulators 120, greater than 12 stimulators 120, less than 12 stimulators 120, and/or other quantities of stimulators 120. Stimulators 120 may be positioned around various portions of cuff 116. For instance, six stimulators 120 may be positioned at a top of cuff 116 and six stimulators 120 may be positioned at a bottom of cuff 116. In some embodiments, cuff 116 may include sets of stimulators 120, such as sets of two, three, and/or other quantities of stimulators 120.

In some embodiments, cuff 116 may include one or more air bladders 132. Air bladder 132 may be made of an expandable and/or stretchable material, such as rubber, plastic, and/or other materials. Air bladder 132 may be positioned within and/or at a side of cuff 116. Air bladder 132 may be positioned over or under one or more stimulators 120. In some embodiments, cuff 116 may include a first air bladder 132 positioned at a top portion of cuff 116 and a second air bladder 132 positioned at a bottom portion of cuff 116.

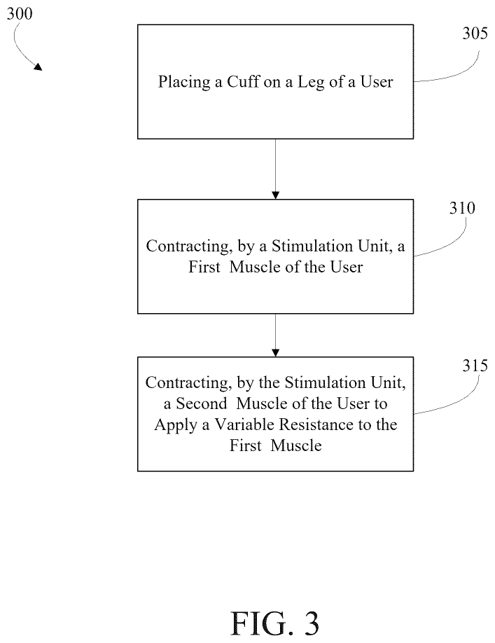

Cuff 116 may include one or more sensors 124. A “sensor” as used in this disclosure is any device capable of detecting physical changes in an object and/or environment. Physical changes may include, but are not limited to, temperatures, pressures, currents, voltages, and the like. For instance, sensor 124 may include current sensors, voltage sensors, hall effect sensors, tactile sensors, temperature sensors, heart rate sensors, respiration rate sensors, and/or other types of sensors. Cuff 116 may include a plurality of sensors 124. A plurality of sensors 124 may include a same sensor type or may be a combination of two or more various sensing devices, such as, but not limited to, a pressure sensor and a current sensor. Cuff 116 may be explained in greater detail below with reference to FIG. 3.

Referring still to FIG. 1, controller 104 may include compression pump 112. Compression pump 112 may be configured to compress air at various pressures and/or volumes. For instance, and without limitation, compression pump 112 include a continuous compression, sequential compression, gradient compression, intermittent compression, cryocompression, or other compression pump device. In some embodiments, compression pump 112 may be a pneumatic or other compression pump. Compression pump 112 may be configured to input ambient air and output compressed air, such as at about 10 pounds per square inch (PSI), greater than 10 PSI, or less than 10 PSI. Compression pump 112 may be in fluidic communication with air bladder 132 of cuff 116. A “fluidic communication” as used in this disclosure is a form of connection between two or more fluidic bodies in which mass of one fluidic body is received at a second fluidic body, and vice versa. For instance and without limitation, compression pump 112 may be in fluidic communication with one or more air bladders 132 through one or more air tubes. An air tube may be a tubing that connects an air outlet of compression pump 112 to an air inlet of air bladder 132. An “air outlet” as used in this disclosure is an exit port of a device in which fluid leaves. An “air inlet” as used in this discourse is an entry port of a device in which fluid enters. Compression pump 112 may provide compressed air at varying PSIs to one or more air bladders 132 through one or more air tubes or other fluidic connection devices. As a non-limiting example, a first air tube may connect a first air outlet of compression pump 112 to an air inlet of a first air bladder 132, and a second air tube may connect a second air outlet of compression pump 112 to an air inlet of a second air bladder 132. In some embodiments, cuff 116 may include two or more air bladders 132, which may be positioned along key areas of cuff 116. Key areas may include, but are not limited to, parts of a leg, parts of an arm, and the like. For instance and without limitation, a key area placement of air bladder 132 on cuff 116 may be at a lower portion of cuff 116 designed to interact with a side of a calf muscle of a user. Cuff 116 may have a first air bladder 132 positioned at a top portion of cuff 116, such as above a knee hole of cuff 116, and a second air bladder 132 positioned at a bottom portion of cuff 116, such as below a knee hole of cuff 116. A knee hole of cuff 116 may be an opening of cuff 116 which may allow movement of a user's knee, such as extension of a user's knee.

In some embodiments, controller 104 may be configured to apply a pressure to air bladder 132 to provide contact between one or more stimulators 120 and a skin of a user. For instance, controller 104 may activate compression pump 112 to inflate air bladder 132, which may cause one or more stimulators 120 to be pushed against a user's skin. A pushing of one or more stimulators 120 against a user's skin by air bladder 132 may increase a contact between a user's skin and a surface area of one or more stimulators 120, which may allow for an increase in current and/or stimulation provided by one or more stimulators 120. In some embodiments, controller 104 may adjust inflation of air bladder 132 automatically based on data generated from sensors 124. For instance, sensors 124 may generate data indicative of a conductivity of a user's skin, which may range from about, but not limited to, 1 microsiemen (μS) to about 100 μS. Based on a conductivity value generated by sensors 124, controller 104 may adjust an inflation of air bladder 132 which may increase a contact of surface area of one or more stimulators 120 and a user's skin. Controller 104 may compare sensor data to one or more skin conductivity values. For instance, if sensor data generated by sensors 124 indicates a skin conductivity value below a threshold skin conductivity value, controller 104 may increase a pressure of air bladder 132 until sensor data generated by sensors 124 reaches the threshold skin conductivity value. Controller 104 may perform an initialization sequence when a user first wears cuff 116 in which conductivity values are determined by data generated from sensors 124 and compared to one or more threshold conductivity values. Controller 104 may increase pressure of air bladder 132 in an initialization sequence until a skin conductivity value threshold is reached. As a non-limiting example, a user may wear cuff 116 for a first time and controller 104 may determine a skin conductivity value of less than about 1 μS, which may be below a skin conductivity value threshold of about 1 μS. Controller 104 may activate compression pump 112 to provide a pressure to air bladder 132 while continuously monitoring skin conductivity values generated by sensors 124. Controller 104 may halt inflation of air bladder 132 once a skin conductivity value determined by sensor data generated by sensors 124 reaches a skin conductivity value threshold. Controller 104 may generate an error signal if contact between stimulators 120 and a user's skin is determined to be poor based on sensor data generated by sensors 124. In some embodiments, a user may adjust inflation of air bladder 132 based on their comfort level through controller 104.

Stimulation unit 108 of controller 104 may be configured to command one or more stimulators 120 to deliver a current, voltage, and the like to a user. Stimulation unit 108 may command one or more stimulators 120 based on sensor data received from sensors 124. Sensor data may include, but is not limited to, voltages, currents, accelerations, and the like. Stimulation unit 108 may be configured to interpret sensor data to determine muscle activity, blood flow, and the like of a limb cuff 116 may be wrapped around. Stimulation unit 108 of control 104 may be configured to command compression pump 112 to apply a pressure to a user's body part via air bladder 132 of cuff 116, in some embodiments. Controller 108 may adjust pressures delivered to air bladder 132 via compression pump 112 based on sensor data received from sensors 124. For instance and without limitation, sensor data may include pressures of one or more air bladders 132. Controller 108 may adjust pressures of air bladder 132 based on sensed pressures of air bladder 132. In some embodiments, controller 108 may be configured to adjust and/or operate stimulators 120 and compression pump 112 in combination, which may deliver a combination of pressures via air bladder 132 and electrical stimulation via stimulators 120 to a user through cuff 116. For instance and without limitation, controller 108 may intermittently increase electrical stimulation via stimulators 120, pressures via air bladders 132, and the like. In some embodiments, controller 108 may increase a pressure of air bladder 132 while decreasing a current of stimulators 120. In other embodiments, controller 108 may increase a current of stimulators 120 while decreasing a pressure of air bladder 132. In some embodiments, controller 108 may increase both a current of stimulators 120 and a pressure of air bladder 132. In some embodiments, controller 108 may decrease a current of stimulators 120 and a pressure of air bladder 132. Controller 108 may be configured to individually operate one or more stimulators 120, air bladders 132, and the like. As a non-limiting example, in 3 sets of two stimulators 120, controller 108 may increase a current of two stimulators 120 while keeping a current unchanged of the rest of the stimulators 120. Likewise, and continuing this non-limiting example, controller 108 may be configured to increase a pressure of a first air bladder 132 while decreasing a pressure of a second air bladder 132. One of ordinary skill in the art, upon reading this disclosure, will appreciate the many various combinations of pressures and electrical stimulations that may be applied via controller 108.

In some embodiments, stimulators 120 may include a first electrode positioned on a front portion of cuff 116. A front portion of cuff 116 may be a surface of cuff 116 that contacts a quadricep and/or shin of a user when cuff 116 is worn. A front portion of cuff 116 may contact other portions of a user's body, such as, but not limited to, a torso, chest, arm, finger, foot, or other muscles. A first electrode may be configured to deliver a current to and/or contract a first muscle of a user. A first muscle of a user may be a quadricep, calf muscle, arm muscle, finger muscle, back muscle, chest muscle, and/or other muscle. A first muscle of a user may be any anterior leg and/or ankle muscle of a user, without limitation. Stimulators 120 may include a second electrode. A second electrode may be positioned on a back portion of cuff 116. A back portion of cuff 116 may be a surface of cuff 116 that contacts a hamstring of a user when cuff 116 is worn. In some embodiments, a back portion of cuff 116 may contact a torso, chest, back, finger, arm, foot, or other portion of a user's body. A second electrode may be configured to deliver a current to and/or contract a second muscle of a user. A second muscle of a user may be a hamstring or any other muscle. A second muscle of a user may be any posterior muscle of a leg or ankle of a user, without limitation. In some embodiments, controller 108 may be configured to activate a first and second electrode simultaneously. For instance, and without limitation, controller 108 may activate both a first electrode configured to contract a first muscle and a second electrode configured to contract a second muscle at a same time as a contraction of the first muscle. Furthering this non-limiting example, controller 108 may contract both a quadricep and hamstring of a user simultaneously. In some embodiments, controller 108 may be configured to provide resistance to one or more muscles of a user through counter-contraction of one or more opposing muscles of the user. In some embodiments, controller 108 may be configured to contract a quadricep while opposing the contraction of the quadricep with a contraction of a hamstring, and vice versa.

Controller 108 may be configured to provide a variable resistance to a muscle of a leg and/or ankle of a user and/or an arm, torso, chest, finger, back, foot, or other muscles of a user. A variable resistance may be provided by co-contraction of a second muscle opposite a contracting first muscle. Resistance may be measured in newtons (N), pounds (lb), kilograms (kg), and the like. As a non-limiting example, a resistance of about 100 N may be applied to a first contracting muscle via a second, opposing contracting muscle. A variable resistance may be adjusted or changed by controller 108 based on a variety of factors, such as, but not limited to, type of muscle contracting, rehabilitation program of a user, history of resistance training of a user, and the like. A variable resistance may change over time, such as in second, minutes, and the like. For instance and without limitation, controller 108 may gradually increase or decrease a resistance applied to a first contracting muscle via increasing or decreasing contraction of a second contracting muscle. In some embodiments, controller 108 may increase a variable resistance over time as a user's muscles adapt to the resistance. Controller 108 may determine an increase in muscle strength of a user's muscle through sensor data of sensor 124. Sensor data of sensor 124 may include acceleration data, force data, gyroscopic data, and the like. A variable resistance may be automatically determined by controller 108 based on sensor data from sensors 124. For instance controller 108 may automatically increase a resistance applied to a first contracting muscle if a force detected by sensor 124 of the first contracting muscle exceeds or falls below one or more thresholds.

Thresholds may include periods of time to reach a specific range of motion, measured force generated by a first contracting muscle, and/or other thresholds. For instance, a threshold may be a range of motion of user's leg of about 20 degrees, and controller 108 may be configured to increase a resistance applied to a quadricep of a user if the user's leg reaches a 20 degree range of motion. A threshold of a force of a contracting muscle may be a specific value, range of values, and the like. As a non-limiting example, a threshold value of a force for a user's quadricep may be about 50 N, which if exceeded controller 108 may increase a resistance applied to the user's quadricep through contraction of a user's hamstring. Thresholds may be calculated automatically by controller 108 based on resistances and measured sensor data of a user. In other embodiments, thresholds may be set by a medical professional, such as part of a recovery program. In other embodiments, a variable resistance may be set by a medical professional based on a recovery plan, treatment plan, and the like. Controller 108 may be configured to operate in a resistance training mode. In a resistance training mode, controller 108 may be configured to contract one or more muscles of a user through one or more repetitions, sets of repetition, and the like at various applied resistances and/or equivalent weights. As a non-limiting example, controller 108 may contract a user's leg muscle to perform three sets of 10 reps of leg extensions with an applied resistance of about 10 N. Controller 108 may automatically adjust a resistance, amount of reps, sets, and the like based on data received from sensors 124. In other embodiments, controller 108 may receive a resistance training program from an external computing device and/or user input.

Resistances may correlate to currents delivered by stimulators 124. For instance, a resistance of 100 N generated by a muscle of a user may correlate to 25 mA of current delivered. In some embodiments, controller 108 may be configured to operate in an initialization mode. An initialization mode may include controller 108 providing various currents through stimulators 120 to a user's leg and measuring a force of contraction of one or more muscles of the user's leg through sensor 124. Controller 108 may be configured to determine a variety of user's current-to-force generation ratio, since each user may generate varying force based on the same applied current. A current-to-force generation ratio may be a ratio of currents to force generated, such as about 1 mA to about 500 N, or ratios greater than or less than about 1 mA to about 500 N. Controller 108 may be configured to correlate currents delivered by stimulators 120 to forces of one or more muscle contractions, by sensor 124. In some embodiments, controller 108 may be configured to determine a current-to-force generation ratio based on sensor data of sensor 124, such as a speed of movement of a user's leg while performing knee or ankle extensions. As a non-limiting example, a user may extend their knee without co-contraction, a speed of which may be measured by sensor 124. Controller 108 may apply incremental co-contraction stimulation until the user's knee does not extend and may determine a range of co-contraction stimulation levels based on the movement of the user's knee and applied currents of stimulators 120.

In some embodiments, controller 108 may utilize a resistance generation machine learning model to determine and/or learn correlations of currents delivered by stimulators 124 to forces of one or more muscle contractions of a user. A resistance generation machine learning model may be trained with training data correlating currents to forces of muscle contractions. Training data may be received through user input, external computing devices, and/or previous iterations of processing. A resistance generation machine learning model may be trained and/or operable to determine estimations of forces generated by a user's muscles based on currents delivered to the user's muscles. In some embodiments, controller 108 may communicate with a remote device that may operate a resistance generation machine learning model and may receive determinations of the resistance generation machine learning model remotely. Controller 108 may be configured to use correlations between currents and forces of muscles generated to determine stimulation parameters of one or more muscles in a user's leg. Stimulation parameters may include currents, voltages, frequencies, peak to peak values, and the like. For instance and without limitation, controller 108 may determine that a current of about 50 mA makes a user's quadricep contract with a force of about 5 lbs. In other embodiments, controller 108 may receive correlations of currents and forces generated by one or more external computing devices, user input, and the like. Controller 108 may be configured to determine muscle fatigue of one or muscles of a user. Muscle fatigue may identified by controller 108 as a diminishing force generated by a user's muscle by a same current value. For instance, controller 108 may determine a decrease in force generated by a user's muscle through sensors 124 while maintaining a current value through stimulators 124. Controller 108 may generate one or more resistance programs based on muscle fatigue, current-to-force generation ratios, and/or other parameters. Controller 108 may generate resistance programs based on progress of a user's muscle development, which may be tracked and/or monitored through one or more sensors 124.

Controller 108 may be configured to move a leg of a user through various ranges of motion by contracting one or more muscles of the leg of the user. Ranges of motion may include degrees, angles, and the like of movement of a leg relative to a hip, knee, ankle, and/or other pivot points. In some embodiments, ranges of motion may include lengths, heights, and/or other distances. As a non-limiting example, a normal range of motion for ankle dorsiflexion may be about 20 degrees from a resting position to a shin of the user and a normal range of motion of knee flexion may be about 150 degrees. A full range of motion may include a full extension of a user's leg, a full retraction of a user's leg, a full extension of a user's ankle, a full retraction of a user's ankle, and/or other ranges of motion. Controller 108 may automatically detect one or more ranges of motion through one or more sensors 124. In some embodiments, controller 108 may act in a feedback loop with sensors 124 and stimulators 120. A feedback loop may include controller 108 adjusting one or more resistances applied to one or more contracting muscles via co-contracting muscles based on sensor data of sensors 124. Sensor data may include angular data, accelerations, gyroscopic data, and/or other data. In other embodiments, controller 108 may be pre-programmed to move a user's leg through various ranges of motion. Pre-programmed ranges of motion may be received by a medical professional, such as a nurse, doctor, or other professional. In some embodiments, controller 108 may utilize a range of motion machine learning model. A range of motion machine learning model may be trained with training data correlating sensor data to ranges of motion. Training data may be received through user input, external computing devices, and/or previous iterations of processing. A range of motion machine learning model may be configured to input sensor data and output various ranges of motion, which may be determined in degrees, radians, heights, lengths, and the like.

Still referring to FIG. 1, in some embodiments, controller 108 may be configured to operate in one or more modes of operation. Modes of operation may include, but are not limited to, automated intermittent pneumatic compression (IPC), exercise, electrical stimulation, and/or other modes. In an IPC mode, controller 108 may be configured to adjust a pressure of one or more air bladders 132, such as in intermittent periods. In an exercise mode, controller 108 may be configured to activate a user's leg muscles through electrical stimulation provided by stimulators 120 while opposing activation of the user's leg muscles through a force provided by co-contracting muscles that may be contracted simultaneously by stimulators 120. In some embodiments, in an exercise mode, compression pump 112 may be deactivated or otherwise shut off. In some embodiments, one or more parameters of an exercise mode may be adjustable. For instance, a nurse or other healthcare provider may select an exercise mode that includes plantar and/or dorsi flexion of an ankle, flexion and/or extension of a knee joint, and/or a combination of both. In some embodiments, one or more parameters of an exercise mode that may be selectable may include repetitions, resistance levels, rest time, sets, durations, and the like. In some embodiments, an exercise mode of controller 108 may include an exercise regime, which may be set by one or more healthcare providers. For instance, an exercise regime may include exercising both legs of a user in an alternating pattern. An electrical stimulation mode of operation may include activating one or more leg muscles of a user. For instance, an electrical stimulation mode of operation may include activating quadriceps, hamstrings, plantar, dorsi, and/or other muscles of a user. In some embodiments, controller 108 may selectively activate one or more stimulators 120 which may selectively activate one or more muscles of a leg of a user. An electrical stimulation mode may include one or more parameters, such as, but not limited to, duration, intensity, and the like. An electrical stimulation mode of operation may be combined with an exercise mode of operation, in some embodiments.

Various information may be displayed through a display device in communication with controller 108. Controller 108 may be in communication with a display device via a wired or wireless connection. Display devices may include, but are not limited to, monitors, smartphones, laptops, tablets, and the like. In some embodiments, controller 108 may be configured to receive user input through one or more user interfaces (UI) of one or more display devices. For instance and without limitation, a user may select one or more modes of operation of controller 108 through a UI of a display device. In some embodiments, controller 108 may display information such as, but not limited to, pressures of compression pump 112 and/or air bladders 132, currents and/or voltage of stimulators 120, an indication of which stimulators 120 and/or air bladders 132 are currently activated, force applied by co-contracting muscles, force generated by a contracting muscle, trends in leg strength of a user, and/or other information.

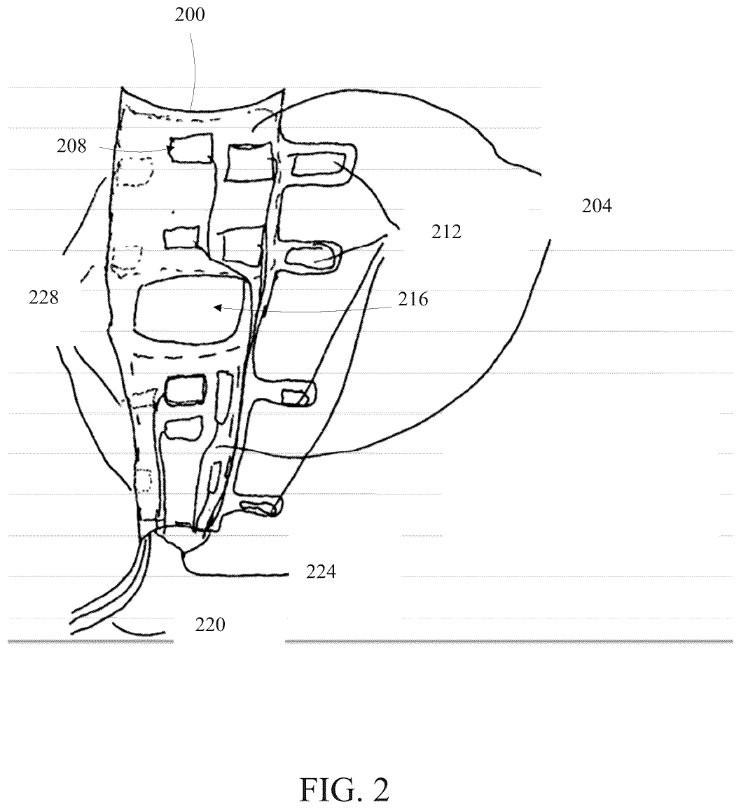

Referring now to FIG. 2, a front view of a cuff 200 is illustrated. Cuff 200 may have air bladders 204 and/or stimulators 208, which may be the same as that of air bladders 132 and stimulators 124 as described above with reference to FIG. 1. In some embodiments, stimulators 208 may be embedded in cuff 200. In an embodiment, cuff 200 may have eight stimulators 208, four in an upper portion of cuff 200 and four in a lower portion of cuff 200. In some embodiments, two stimulators 208 may be positioned over quadricep muscles of a user and two stimulators 208 may be positioned over a hamstring of a user. In a lower portion of cuff 200, in some embodiments, two stimulators 208 may be positioned over a tibialis anterior of a patient and another two stimulators 208 may be positioned over a gastrocnemius of the patient. In some embodiments, each stimulator 208 may be connected to one another via one or more wires, leads, and the like, without limitation. Stimulators 208 may be grouped into sets, such as sets of two or more stimulators 208. In some embodiments, sets of stimulators 208 may have their own electrical channel which may be in communication with a controller or other device. As a non-limiting example, the two stimulators 208 positioned over the gastrocnemius of the patient described above may have a separate electrical channel than that of the two stimulators 208 positioned over the tibialis anterior of the patient.

In some embodiments, cuff 200 may be attachable to a user via attachment tabs 212. Attachment tabs 212 may include Velcro, hooks, loops, clips, and/or other attachment devices. In some embodiments, cuff 200 may include four attachment tabs 212 or greater or less than four attachment tabs 212. Attachment tabs 212 may wrap around cuff 200 and attach to a surface on an opposite side of attachment tabs 212, which may help secure cuff 200 to a user's limb. Attachment tabs 212 may be configured to attach to attachment surfaces 228. Attachment surfaces 228 may be Velcro, in some embodiments. In some embodiments, cuff 200 may include knee hole 216. K nee hole 216 may be a cut-out of cuff 200, in some embodiments. For instance, knee hole 216 may allow for a user's knee to extend and retract while cuff 200 is worn by a user's leg. Cuff 200 may have stimulators 208 above and/or below knee hole 216. In some embodiments, cuff 200 may have air bladders 204 above and/or below knee hole 216.

Cuff 200 may include air tubes 220. Air tubes 220 may be in fluidic communication with one or more air bladders 204 of cuff 200. Air tube 220 may be configured to attach to an air outlet of a compression pump, such as described above with reference to FIGS. 1-2. In some embodiments, cuff 200 may have three or more air tubes 220. In other embodiments, cuff 200 may have less than three air tubes 220. Cuff 200 may include leads 224. Leads 224 may be electrically conductive material that may provide a connection to one or more stimulators 204. Leads 224 may be connectable to one or more external power supplies. In some embodiments, leads 224 may provide current, voltage, and the like to one or more stimulators 208.

Referring now to FIG. 3, a flowchart 300 of a method of preventing permanent atrophy and/or DVT edema is presented. At step 305, method 300 includes placing a cuff on a leg of a user. A cuff may include an electrode configured to stimulate a portion of the leg of the user. In some embodiments, a cuff may include a plurality of electrodes configured to stimulate various portions of the leg of the user. A cuff may have a first electrode positioned on a front surface of the cuff and a second electrode positioned on a back surface of the cuff. In some embodiments, a cuff may include an air bladder configured to apply pressure to a portion of a leg of a user. An air bladder may be made of a flexible material, such as plastic or another material, without limitation. A cuff may include two or more air bladders. In some embodiments, a cuff may include an air inlet port fluidically connected to an air bladder. An air inlet port may be configured to connect to one or more external fluid sources. In some embodiments, a cuff may have a first air bladder positioned at a top portion of the cuff and a second air bladder positioned at a bottom portion of the cuff. This step may be implemented, without limitation, as described above with reference to FIGS. 1-2.

At step 310, method 300 includes contracting, by a stimulation unit, a first muscle of the user. A first muscle may be a quadricep, hamstring, ankle muscle, or other muscle. Contraction may include delivering a current to a muscle of the user through an electrode. A controller of a stimulation unit may determine a current-to-force generation ratio and apply a current based on this ratio. This step may be implemented, without limitation, as described above with reference to FIGS. 1-2.

At step 315, method 300 includes contracting, by the stimulation unit, a second muscle of the user to apply a variable resistance to the first muscle. Contracting a second muscle may include delivering a current to the second muscle via one or more electrodes. A second muscle may be contract simultaneously as the first muscle in step 310. A co-contraction of the second muscle may oppose a contraction of the first muscle, which may provide a resistance. A controller of a stimulation unit may adjust various resistances applied to the first muscle of the user through contraction of the second muscle of the user. In some embodiments, a controller of a stimulation unit may act in a feedback loop in connection with one or more sensors of the cuff. For instance, a controller of the stimulation unit may receive force data, acceleration data, gyroscopic data, and the like and adjust a variable resistance of the first muscle based on various forms of data. This step may be implemented, without limitation, as described above with reference to FIGS. 1-2.

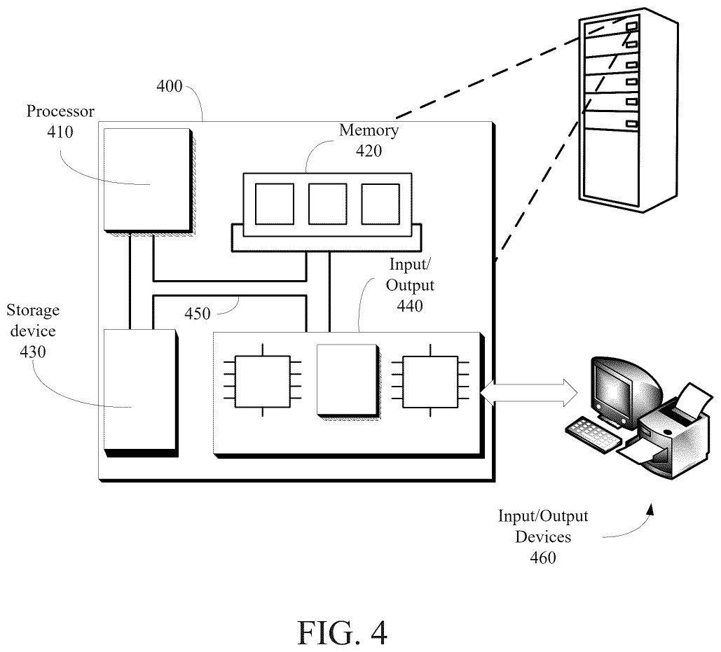

FIG. 4 is a block diagram of an example computer system 400 that may be used in implementing the technology described in this document. General-purpose computers, network appliances, mobile devices, or other electronic systems may also include at least portions of the system 400. The system 400 includes a processor 410, a memory 420, a storage device 430, and an input/output device 440. The apparatus may include disk storage and/or internal memory, each of which may be communicatively connected to each other. The apparatus 100 may include a processor 410. The processor 410 may enable both generic operating system (OS) functionality and/or application operations. In some embodiments, the processor 410 and the memory 420 may be communicatively connected. As used in this disclosure, “communicatively connected” means connected by way of a connection, attachment, or linkage between two or more elements which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

In some embodiments, the processor 410 may include any computing device as described in this disclosure, including without limitation a microcontroller, microprocessor, digital signal processor (DSP) and/or system on a chip (SoC) as described in this disclosure. The processor 410 may include, be included in, and/or communicate with a mobile device such as a mobile telephone or smartphone. The processor 410 may include a single computing device operating independently, or may include two or more computing device operating in concert, in parallel, sequentially or the like. Two or more computing devices may be included together in a single computing device or in two or more computing devices.

The processor 410 may interface or communicate with one or more additional devices as described below in further detail via a network interface device. Network interface device may be utilized for connecting the processor 410 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device.

The processor 410 may include but is not limited to, for example, a computing device or cluster of computing devices in a first location and a second computing device or cluster of computing devices in a second location. The processor 410 may include one or more computing devices dedicated to data storage, security, distribution of traffic for load balancing, and the like. The processor 410 may distribute one or more computing tasks as described below across a plurality of computing devices of computing device, which may operate in parallel, in series, redundantly, or in any other manner used for distribution of tasks or memory between computing devices. The processor 410 may be implemented using a “shared nothing” architecture in which data is cached at the worker, in an embodiment, this may enable scalability of system 400 and/or processor 410.

With continued reference to FIG. 4, processor 410 and/or a computing device may be designed and/or configured by memory 420 to perform any method, method step, or sequence of method steps in any embodiment described in this disclosure, in any order and with any degree of repetition. For instance, the processor 410 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. The processor 410 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Each of the components 410, 420, 430, and 440 may be interconnected, for example, using a system bus 450. The processor 410 is capable of processing instructions for execution within the system 400. In some implementations, the processor 410 is a single-threaded processor. In some implementations, the processor 410 is a multi-threaded processor. In some implementations, the processor 410 is a programmable (or reprogrammable) general purpose microprocessor or microcontroller. The processor 410 is capable of processing instructions stored in the memory 420 or on the storage device 430.

The memory 420 stores information within the system 400. In some implementations, the memory 420 is a non-transitory computer-readable medium. In some implementations, the memory 420 is a volatile memory unit. In some implementations, the memory 420 is a non-volatile memory unit.

The storage device 430 is capable of providing mass storage for the system 400. In some implementations, the storage device 430 is a non-transitory computer-readable medium. In various different implementations, the storage device 430 may include, for example, a hard disk device, an optical disk device, a solid-date drive, a flash drive, or some other large capacity storage device. For example, the storage device may store long-term data (e.g., database data, file system data, etc.). The input/output device 440 provides input/output operations for the system 400. In some implementations, the input/output device 440 may include one or more network interface devices, e.g., an Ethernet card, a serial communication device, e.g., an RS-232 port, and/or a wireless interface device, e.g., an 802.11 card, a 3G wireless modem, or a 4G/5G wireless modem. In some implementations, the input/output device may include driver devices configured to receive input data and send output data to other input/output devices, e.g., keyboard, printer and display devices 460. In some examples, mobile computing devices, mobile communication devices, and other devices may be used.

In some implementations, at least a portion of the approaches described above may be realized by instructions that upon execution cause one or more processing devices to carry out the processes and functions described above. Such instructions may include, for example, interpreted instructions such as script instructions, or executable code, or other instructions stored in a non-transitory computer readable medium. The storage device 430 may be implemented in a distributed way over a network, for example as a server farm or a set of widely distributed servers, or may be implemented in a single computing device.

Although an example processing system has been described in FIG. 4, embodiments of the subject matter, functional operations and processes described in this specification can be implemented in other types of digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible nonvolatile program carrier for execution by, or to control the operation of, a data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

A user may also input commands and/or other information to computer system 400 via storage device 424 (e.g., a removable disk drive, a flash drive, etc.) and/or network interface device 440. A network interface device, such as network interface device 440, may be utilized for connecting computer system 400 to one or more of a variety of networks, such as network 444, and one or more remote devices 448 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 444, may employ a wired and/or a wireless mode of communication. In general, any network topology may be used. Information (e.g., data, software 420, etc.) may be communicated to and/or from computer system 400 via network interface device 440.

Computer system 400 may further include a video display adapter 452 for communicating a displayable image to a display device, such as display device 436. Examples of a display device include, but are not limited to, a liquid crystal display (LCD), a cathode ray tube (CRT), a plasma display, a light emitting diode (LED) display, and any combinations thereof. Display adapter 452 and display device 436 may be utilized in combination with processor 404 to provide graphical representations of aspects of the present disclosure. In addition to a display device, computer system 400 may include one or more other peripheral output devices including, but not limited to, an audio speaker, a printer, and any combinations thereof. Such peripheral output devices may be connected to bus 412 via a peripheral interface 456. Examples of a peripheral interface include, but are not limited to, a serial port, a USB connection, a FIREWIRE connection, a parallel connection, and any combinations thereof.

The terms “about” or “substantially” that modify a condition or relationship characteristic of a feature or features of an embodiment of the disclosure, are to be understood to mean that the condition or characteristic is defined to within tolerances that are acceptable for operation of the embodiment for an application for which it is intended. For example, the terms “about,” “substantially,” and/or “close” with respect to a magnitude or a numerical value may imply to be within an inclusive range of −10% to +10% of the respective magnitude or value.

It must be noted that, as used in the specification, the singular forms “a,” “an” and “the” include plural referents unless the context clearly dictates otherwise. By way of example, “an analogue” means one analogue or more than one analogue.

Each numerical value presented herein is contemplated to represent a minimum value or a maximum value in a range for a corresponding parameter. Accordingly, when added to the claims, the numerical value provides express support for claiming the range, which may lie above or below the numerical value, in accordance with the teachings herein. Every value between the minimum value and the maximum value within each numerical range presented herein (including in the figures), is contemplated and expressly supported herein, subject to the number of significant digits expressed in each particular range. Absent express inclusion in the claims, each numerical value presented herein is not to be considered limiting in any regard.

Having described certain embodiments of the disclosure, it will be apparent to those of ordinary skill in the art that other embodiments incorporating the concepts disclosed herein may be used without departing from the spirit and scope of the disclosure. Accordingly, the described embodiments are to be considered in all respects as only illustrative and not restrictive. The terms and expressions employed herein are used as terms and expressions of description and not of limitation and there is no intention, in the use of such terms and expressions, of excluding any equivalents of the features shown and described or portions thereof. The structural features and functions of the various embodiments may be arranged in various combinations and permutations, and all are considered to be within the scope of the disclosure. Unless otherwise necessitated, recited steps in the various methods may be performed in any order and certain steps may be performed substantially simultaneously and/or in parallel.

1. A system for prevention of permanent atrophy and/or deep-vein thrombosis (DVT) edema, comprising:
a cuff, the cuff comprising:
a first electrode positioned on a front portion of the cuff, the first electrode configured to contract a first muscle of a user;
a second electrode positioned on a back portion of the cuff, the back portion opposite the front portion, wherein the second electrode is configured to contract a second muscle of a user; and
a controller in electrical communication with the cuff, the controller comprising:
a stimulation unit, wherein the stimulation unit is configured to activate the first and second electrodes to simultaneously contract the first muscle of the user while providing a variable resistance to the first muscle of the user through contraction of the second muscle of the user.

2. The system of claim 1, wherein the first muscle of the user is a quadricep muscle and the second muscle of the user is a hamstring muscle.

3. The system of claim 1, wherein the cuff further comprises a sensor in communication with the stimulation unit, wherein the controller is further configured to adjust the variable resistance based on sensor data received from the sensor of the cuff in a feedback loop.

4. The system of claim 1, wherein the stimulation unit is further configured to simultaneously contract the second muscle of the user while providing a variable resistance to the second muscle of the user through a contraction of the first muscle of the user.

5. The system of claim 1, wherein the stimulation unit is further configured to move a leg of the user through a range of motion by contracting either the first muscle or the second muscle of the user.

6. The system of claim 5, wherein the stimulation unit is further configured to adjust the range of motion based on sensor data received from a sensor of the cuff.

7. The system of claim 1, wherein the stimulation unit is further configured to provide a stimulation pattern to a leg of the user through the first and second electrodes.

8. The system of claim 1, wherein the cuff further comprises:
an air bladder configured to apply pressure to a portion of a leg of a user; and
an air inlet port fluidically connected to the air bladder.

9. The system of claim 8, wherein the stimulation unit is further configured to provide air pressure to the air bladder through activation of an air pump of the stimulation unit, the air pump fluidically connected to the air inlet port of the air bladder.

10. The system of claim 1, wherein the stimulation unit is further configured to increase or decrease the variable resistance in time intervals.

11. A method for prevention of permanent atrophy and/or deep-vein thrombosis (DVT) edema, comprising:
placing a cuff on a leg of a user, the cuff comprising:
a first electrode positioned on a front portion of the cuff, the first electrode configured to contract a first muscle of the user;
a second electrode positioned on a back portion of the cuff, the back portion opposite the front portion, wherein the second electrode is configured to contract a second muscle of the user; and
contracting, by a stimulation unit in communication with the first and second electrodes, a first muscle of the user through the first electrode;
contracting, by the stimulation unit, a second muscle of the user through the second electrode to apply a variable resistance to the first muscle of the user, wherein contraction of the first and second muscles occur simultaneously.

12. The method of claim 11, wherein the first muscle of the user is a quadricep muscle and the second muscle of the user is a hamstring muscle.

13. The method of claim 11, further comprising adjusting, by the stimulation unit, the variable resistance based on sensor data received from a sensor of the cuff in a feedback loop.

14. The method of claim 11, further comprising contracting the second muscle of the user while providing a variable resistance to the second muscle of the user through a contraction of the first muscle of the user.

15. The method of claim 11, further comprising moving a leg of the user through a range of motion by contracting either the first muscle or the second muscle of the user.

16. The method of claim 15, further comprising adjusting the range of motion by the stimulation unit based on sensor data received from a sensor of the cuff.

17. The method of claim 11, further comprising providing a stimulation pattern to the leg of the user through the first and second electrodes.

18. The method of claim 11, wherein the cuff further comprises:
an air bladder configured to apply pressure to a portion of a leg of a user; and
an air inlet port fluidically connected to the air bladder.

19. The method of claim 18, further comprising providing air pressure to the air bladder through activation of an air pump of the stimulation unit, the air pump fluidically connected to the air inlet port of the air bladder.

20. The method of claim 11, further comprising increasing or decreasing the variable resistance applied to the first muscle of the user in time intervals.
